(19) United States
(12) Patent Application Publication
(10) Pub. No.: US 2022/0387513 A1
(43) Pub. Date: Dec. 8, 2022

(54) COMPOSITIONS AND METHODS FOR ENHANCING RETINAL GANGLION CELL DEVELOPMENT AND PLURIPOTENT STEM CELL-DERIVED THREE-DIMENSIONAL TISSUE

(71) Applicant: THE TRUSTEES OF INDIANA UNIVERSITY, Bloomington, IN (US)

(72) Inventors: Jason S. Meyer, Whitestown, IN (US); Clarisse Fligor, Indianapolis, IN (US)

(21) Appl. No.: 17/770,183

(22) PCT Filed: Oct. 21, 2020
(86) PCT No.: PCT/US2020/056624

§ 371 Date: Apr. 19, 2022

Related U.S. Application Data
(60) Provisional application No. 62/923,886, filed on Oct. 21, 2019.

(51) Int. Cl. A61K 35/30; A61P 27/02

(52) U.S. Cl. CPC A61K 35/30; A61P 27/02

(57) ABSTRACT

Various aspects and embodiments disclosed herein relate generally to the stem cell biology and cell replacement therapy. Embodiments include compositions and methods for modelling, treatment, reducing resistance to the treatment, prevention, and diagnosis of a condition/disease associated with retinal degenerative diseases or a related clinical condition thereof. Other embodiments include methods and compositions for developing pluripotent stem cell-derived 3D tissues.

### CROSS-REFERENCE TO RELATED APPLICATIONS

This application claims the benefit of U.S. Provisional Application No. 62/923,886, filed Oct. 21, 2019, the entire disclosure of which is hereby expressly incorporated by reference herein.

### STATEMENT OF GOVERNMENTAL RIGHTS

This invention was made with government support under EY024984 awarded by National Institutes of Health. The government has certain rights in the invention.

### FIELD

Various aspects and embodiments disclosed herein relate generally to the stem cell and cell replacement therapy. Embodiments include compositions and methods for modelling, treatment, reducing resistance to the treatment, prevention, and diagnosis of a condition/disease associated with retinal degenerative diseases or a related clinical condition thereof. Other embodiments include methods and compositions for developing pluripotent stem cell-derived 3D tissues (e.g., organoids and/or assembloids).

### BACKGROUND

Retinal organoids are three-dimensional structures derived from human pluripotent stem cells (hPSCs) which mimic the spatial and temporal differentiation of the retina, that can be served as effective models of retinal development in vitro. Retinal ganglion cells (RGCs) can be developed and organized within retinal organoids.

Retinal ganglion cells (RGCs) play a critical role in the transmission of visual information between the eye and the brain, and thus, a damage and loss of RGC axons can lead to many retinal degenerative diseases. As RGCs have a limited capacity for regeneration following damages, it has been limited by many obstacles including an inability to regrow long-distance connections. Additionally, at later stages of RGC degeneration following cell death, a need exists to replace the large number of RGCs.

Human pluripotent stem cells (hPSCs), including both embryonic and induced pluripotent stem cells, are attractive candidates for translational approaches, due to their ability to divide indefinitely as well as differentiate into any cell type in the body. Recent studies have demonstrated the ability to differentiate hPSCs into RGCs. However, the RGCs that were derived in a stochastic manner often lacks the organization typical of the retina, including the cell-to-cell interactions associated with retinogenesis. In part, due to the inability to extend axons across long distances as well as the lack of capacity to appropriately respond to extrinsic guidance cues to regulate this outgrowth, their ability to serve as a model of retinal development is limited, as well as their utility for cell replacement therapies. Thus, a need exists for the development of an in vitro system that facilitates differentiation of retinal organoids in a manner that closely mimics the spatial and temporal development of RGCs. This would provide a superior and more representative model of RGC development, facilitating applications of hPSC-derived RGCs for disease modeling, drug screening, as well as cell replacement.

### SUMMARY

Embodiments of the instant application relate to the stem cell biology and cell replacement therapy. Embodiments include compositions and methods for modelling, treatment, reducing resistance to the treatment, prevention, and diagnosis of a condition/disease associated with retinal degenerative diseases or a related clinical condition thereof. Other embodiments include methods and compositions for developing pluripotent stem cell-derived 3D tissues (e.g., organoids and/or assembloids) and retinal ganglion cells with elongated axons.

A first embodiment includes a three-dimensional neural tissue composition, comprising: an assembloid comprising two or more region-specific organoids, comprising: at least one retinal organoid; at least one cortical organoid; at least one thalamic organoid; and at least one other region-specific organoid that does not recapitulate the development of retina, cortex, or thalamus, wherein the two or more region-specific organoids are operatively fused to form the assembloid.

A second embodiment includes the composition according to the first embodiment, wherein the assembloid comprises the at least one retinal organoid and the at least one cortical organoid.

A third embodiment includes the composition according to any one of the first and the second embodiments, wherein the assembloid comprises the at least one retinal organoid, the at least one cortical organoid, and the at least one thalamic organoid.

A fourth embodiment includes the composition according to any one of the first to the third embodiments, wherein the at least one cortical organoid is fused directly to the at least one retinal organoid and/or the at least one thalamic organoid is fused directly to the at least one retinal organoid.

A fifth embodiment includes the composition according to any one of the first to the fourth embodiments, wherein a first end of the at least one thalamic organoid is fused directly to the at least one retinal organoid and a second end of the at least one thalamic organoid is fused directly to the at least one cortical organoid or wherein a first end of the at least one cortical organoid is fused directly to the at least one retinal organoid and a second end of the at least one cortical organoid is fused directly to the at least one thalamic organoid or wherein the at least one retinal organoid, the at least one cortical organoid, and the at least one thalamic organoid are directly and/or indirectly fused together.

A sixth embodiment includes the composition of any one of the first to the fifth embodiments, the composition further comprises retinal ganglion cells or progenitors thereof residing in the at least one retinal organoid, the at least one cortical organoid, and/or the at least one thalamic organoid.

A seventh embodiment includes the composition of any one of the first to the sixth embodiments, wherein the retinal ganglion cells have elongated axons (RGC axons) extending into the at least one thalamic organoid and/or the at least one cortical organoid, and/or wherein a greater number of the RGC axons extend into the at least one thalamic organoid when compared to the number of the RGC axons extend into the at least one cortical organoid, and/or wherein the assembloid further comprises the retinal ganglion cells exhibiting extensive axonal outgrowth compared to the retinal ganglion cells grown in the at least retinal organoid alone, and/or wherein the retinal ganglion cells comprises 2, 3, 4, 5, 6, 7, 8, 10, 11, 12, 13, 14, 15, 20 times longer RGC axons compared to that of the retinal ganglion cells grown in the at least retinal organoid alone.

An eighth embodiment includes the composition of any one of the first to the seventh embodiments, the composition further comprises thalamic cells in the at least one thalamic organoid that have migrated into the at least one retinal organoid, and/or further comprises thalamic cells in the at least one retinal organoid and/or the at least one thalamic organoid.

A ninth embodiment includes the composition of any one of the first to the eighth embodiments, each of the at least one retinal organoid, the at least one cortical organoid, and the at least one thalamic organoid is derived from pluripotent stem cells.

A tenth embodiment includes the composition of any one of the first to the ninth embodiments, the assembloid further comprises the highly proliferative retinal ganglion cells (RGC) compared to the retinal ganglion cells (RGC) grown in the at least retinal organoid alone, and/or the assembloid further comprises 2, 3, 4, 5, 6, 7, 8, 10, 11, 12, 13, 14, 15, 20 times more retinal ganglion cells (RGC) compared to the number of the retinal ganglion cells (RGC) grown in the at least retinal organoid alone.

An eleventh embodiment includes a method of generating retinal ganglion cells (RGC) with elongated axons, comprising the steps of: generating the assembloid according to any one of the first to the tenth embodiments; and isolating retinal ganglion cells (RGC).

A twelfth embodiment includes the method of the eleventh embodiment, the method further comprising: differentiating pluripotent stem cells into at least one region-specific organoid, comprising: at least one retinal organoid; at least one cortical organoid; at least one thalamic organoid; and at least one other region-specific organoid that does not recapitulate the development of retina, cortex, or thalamus.

A thirteenth embodiment includes the method according to any one of the eleventh and the twelfth embodiments, further comprising: subjecting pluripotent stem cells to floating culture to induce differentiation into retinal progenitor cells.

A fourteenth embodiment includes the method according to any one of the eleventh to the thirteenth embodiments, further comprising: developing the at least one region-specific organoid separately; and fusing two or more region-specific organoids together or allowing two or more region-specific organoids to fuse together.

A fifteenth embodiment includes the method of any one of the eleventh to the fourteenth embodiments, further comprising: allowing axons to elongate into region-specific organoids other than the retinal organoid or allowing axons to elongate into the at least one at least one cortical organoid and/or at least one thalamic organoid.

A sixteenth embodiment includes the method according to any one of the eleventh to the fifteenth embodiments, wherein each of the at least one region-specific organoid is developed separately for about 10, 20, 30, 40, 50, 60, 70, 80, 90, 100, and/or 125 days prior to fusion and fused for about 1, 2, 3, 4, 5, 6, 7, 8, 9, 10, 20, 30, 40, 50, 60, 70, 80, 90, 100 days, or 1-10 days, 1-20 days, 1-30 days, 1-40 days, 1-50 days, 3-10 days, 3-20 days, 3-30 days, 5-10 days, 5-20 days, and/or 5-30 days, or any combination thereof.

A seventeenth embodiment includes the method according to any one of the eleventh to the sixteenth embodiments, the pluripotent stem cells comprise embryonic stem cells and/or induced pluripotent stem cells. In some embodiments, the pluripotent stem cells are derived from a human or an animal.

An eighteenth embodiment includes a therapeutic composition comprising a plurality of retinal ganglion cells with elongated axons obtained by the methods according to any one of the eleventh to the seventeenth embodiments and/or the composition according to any one of the first to the tenth embodiments.

A nineteenth embodiment includes a method for treating a retinal degenerative disease, comprising: administering to a subject a therapeutically effective amount of the therapeutic composition according to the eighteenth embodiment, wherein the subject is diagnosed with a retinal degenerative disease or a related condition thereof.

A twentieth embodiment includes the method according to the nineteenth embodiment, wherein the retinal degenerative disease or a related condition thereof includes age-related macular degeneration (AMD), retinitis pigmentosa (RP), glaucomatous diseases, hereditary optic neuropathy, optic nerve hypoplasia, ischemic disorders, and retinal diseases. Specific examples may include, but are not limited to, glaucoma (e.g., glaucomatous constriction of the visual field and glaucomatous atrophy of the optic nerve), autosomal dominant atrophy of the optic nerve, Leber's hereditary optic neuropathy (Leber's disease), idiopathic optic neuritis, optic nerve hypoplasia involved with iridosteresis, optic neuromyelitis (demyelination), multiple sclerosis (demyelination), ischemic optic neuropathy, central retinal artery occlusion, branch retinal artery occlusion, central retinal vein occlusion, branch retinal vein occlusion, traumatic or drug-induced optic neuropathy, diabetic optic neuropathy, retinopathy of prematurity, and retinal detachment, and/or wherein the subject is a human or an animal.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1 shows differentiation of retinal and cortical organoids. hPSCs were differentiated following established protocols that generates two populations of organoids. Retinal organoids are identified by their bright outer ring as well as a BRN3:TdTomato red fluorescent reporter. Cortical organoids develop along side retinal organoids and contain neural rosette structures throughout.

FIG. 2 shows generation of retina-cortical assembloids. RGCs could be readily identified by TdTomato expression observed in the inner layers of each organoid, defining the presumptive retinal ganglion cell layer. To create retina-cortical assembloids retinal organoids are allowed to fuse with cortical organoids for three days. The BRN3:TdTomato reporter allows for visualization of RGC neurites extending into cortical organoids. Generation of retinal-cortical assembloids more accurately models in vivo architecture of the human diencephalon, composed of the forebrain and developing eyecup.

FIGS. 3A-3G show generation of two genetically distinct populations of organoids. (a-c) RGCs begin to develop in the innermost layer of retinal organoids around 30 days of differentiation, followed by the development of a distinctly separate photoreceptor layer by 70 days of differentiation. (d-f) Cortical organoids contain rosettes enriched with progenitors, similar to the ventricular zone. Earlier born CTIP2+ neurons reside outside of the ventricular zone and later born SATB2+ neurons begin to migrate to a separate outer layer within cortical organoids. (g) Both organoid populations are of neural origin, however, retinal organoids express retinal markers while cortical organoids express cortical markers.

FIGS. 4A-4D show retina-cortical assembloids mimic in vivo architecture. (a-b) Retinal and cortical organoids can be fused to form retina-cortical assembloids. BRN3:TdTomato RGCs extend axons into cortical organoids. (c-d) Growth cones at the leading edge of RGC axons confirms RGCs are actively growing into cortical neurons, suggesting the cortical environment supports RGC outgrowth.

FIGS. 5A-5H show that assembloids significantly increase retinal area and proliferation. (a-f) Retinal organoids grown alone were compared to retinal organoids fused to cortical organoids after 3, 5, and 7 days post fusion (dpf). (g) After 3 days of fusing with cortical organoids (53 days total growth) retinal area of retina-cortical assembloids was significantly increased compared to control retinal organoids. (h) Retina-cortical assembloids displayed a significant increase in cell proliferation by 55 days of total differentiation when compared to controls.

FIGS. 6A-6F show that long term retina-cortical assembloids maintain RGC populations. BRN3:TdTomato expression begins to decrease in control organoids after 100 days in culture.

FIGS. 7A-7N show that long term retina-cortical assembloids maintain RGC populations. (a-h) Retinal organoids were maintained up to 150 days in culture and compared to age matched retina-cortical assembloids. (i-k) Retinal area was significantly increased in assembloids. (1-n) BRN3:TdTomato expression begins to decrease in control organoids after 100 days in culture, while expression is significantly increased in assembloids and continues to increase over time.

FIGS. 8A-81I show that RGC axons actively grow into assembloids and avoid ventricular zone. (a) Retinal organoids expressing BRN3:TdTomato are fused to CTIP2+ cortical organoids at 50 days of differentiation. (b) schematic view of method for quantifying axonal outgrowth. (c) Quantification of range index at 3, 5, and 7 days post fusion (dpf). (d-f) Representative images of outgrowth at 3 dpf (d) 5 dpf (e) and 7 dpf (f). (g-h) RGC axons display outgrowth abilities by extending into cortical organoids. RGC axons also display pathfinding abilities by avoiding SOX2+ ventricular-like zones within cortical organoids.

FIGS. 9A-9J show visual pathway reconstruction with retinal, thalamic, and cortical organoids. (A) Retinal, thalamic and cortical organoids were fused together for to generate Retino-Thalamo-Cortical triassembloids. (B) Fluorescent reporters were used to identify various organoids and their projections with retinal organoids expressing a BRN3:tdTomato reporter and thalamic organoids expressing a GFP reporter. (C-D) Within one week following the fusion of organoids to form assembloids, tdTomato-expressing retinal ganglion cell axons were found to robustly extend into GFP-expressing thalamic organoids. (E) Significantly greater numbers of RGC axons had extended into thalamic organoids compared to cortical organoids at the same time point. (F-H) After an additional 2 months of differentiation, GFP-expressing thalamic cells were found migrating retrogradely into retinal organoids, with these migratory cells expressing the early astrocyte marker S100β. (I-J) On the other side of these assembloids, robust extension of GFP-expressing neurites was observed entering CTIP2-positive cortical organoids. Error bars represent S.E.M., *** p<0.005.

### DEFINITIONS

“About” refers to a range of values plus or minus 10 percent, e.g. about 1.0 encompasses values from 0.9 to 1.1.

“Assembloids” refer to self-organizing three-dimensional miniature organs grown in vitro made by combining two or more organoids resembling distinct areas that can be used to model aspects of interactions that occur in a subject.

“Cortical organoids (COs)” refer to self-organizing three-dimensional miniature organs grown in vitro that model features of the developing human cerebral cortex. Cortical organoids are created by culturing human pluripotent stem cells in a three-dimensional rotational bioreactor and develop over a course of months.

“Embryonic stem cells”, “ES cells” or “ESCs” refer to pluripotent stem cells derived from early embryos.

“Induced pluripotent stem cells,” “iPS cells” or “iPSCs” refer to a type of pluripotent stem cell that has been prepared from a non-pluripotent cell, such as, for example, an adult somatic cell, or a terminally differentiated cell, such as, for example, a fibroblast, a hematopoietic cell, a myocyte, a neuron, an epidermal cell, or the like, by introducing into the non-pluripotent cell or contacting the non-pluripotent cell with one or more reprogramming factors.

“Organoid” refers to a tiny, self-organized three-dimensional multicellular in vitro tissue construct that mimics its corresponding in vivo organ, such that it can be used to study aspects of that organ in the tissue culture dish. An organoid is derived from stem cells and it can be crafted to replicate much of the complexity of an organ, or to express selected aspects of it like producing only certain types of cells.

“Pluripotent stem cell” or “PSCs” refers to a cell that has the potential to differentiate into any cell type, for example, cells of any one of the three germ layers: endoderm, mesoderm, or ectoderm.

“Retinal degenerative disease or a condition thereof” includes, but is not limited to, vision loss, complete blindness, age-related macular degeneration (AMD), and retinitis pigmentosa (RP). Also, it includes any disease that is related to the damage and loss of RGC axons. Examples of retinal degenerative disease involving retinal ganglion cell damage may include glaucomatous diseases, hereditary optic neuropathy, optic nerve hypoplasia, ischemic disorders, and retinal diseases. Specific examples may include, but are not limited to, glaucoma (e.g., glaucomatous constriction of the visual field and glaucomatous atrophy of the optic nerve), autosomal dominant atrophy of the optic nerve, Leber's hereditary optic neuropathy (Leber's disease), idiopathic optic neuritis, optic nerve hypoplasia involved with iridosteresis, optic neuromyelitis (demyelination), multiple sclerosis (demyelination), ischemic optic neuropathy, central retinal artery occlusion, branch retinal artery occlusion, central retinal vein occlusion, branch retinal vein occlusion, traumatic or drug-induced optic neuropathy, diabetic optic neuropathy, retinopathy of prematurity, and retinal detachment.

“Retinal organoids (ROs)” refer to three-dimensional structures derived from pluripotent stem cells (e.g., human PSCs) which recapitulate the spatial and temporal differentiation of the retina, serving as effective in vitro models of retinal development.

“Subject” refers to a mammal or a human.

“Thalamic organoids” refer to three-dimensional structures derived from pluripotent stem cells (e.g., human PSCs) which recapitulate the development of thalamus.

“Therapeutically effective dose” or “therapeutically effective amount” refers to a dose or amount that provides effective treatment of a disease or disorder in a subject. A therapeutically effective dose can vary from compound to compound, from cell to cell, and from subject to subject, and can depend upon factors such as the condition of the subject, the route of delivery, the disease and/or symptoms of the disease, severity of the disease and/or symptoms of the disease or disorder, the age, weight, and/or health of the subject to be treated, and the judgment of the prescribing physician.

“Treat,” “treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treat,” “treating” or “treatment” also refers to inhibiting the disease, either physically, (e.g., stabilization of a discernible symptom), physiologically, (e.g., stabilization of a physical parameter), or both, and to inhibiting at least one physical parameter that can or cannot be discernible to the subject. In certain embodiments, “treat,” “treating” or “treatment” refers to delaying the onset of the disease or at least one or more symptoms thereof in a subject which can be exposed to or predisposed to a disease even though that subject does not yet experience or display symptoms of the disease.

### DETAILED DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

Human pluripotent stem cells possess the remarkable ability to self-organize and differentiate into three dimensional structures known as organoids, which recapitulate development and function of the human brain. Region-specific organoids can be developed and assembled to model complex cell-cell interactions. Generation of retinal-cortical assembloids more accurately models in vivo architecture of the human diencephalon, composed of the forebrain and developing eyecup. As projection neurons of the retina, retinal ganglion cells (RGCs) serve a vital role in vision, with their axons creating a vital link between the eye and the brain. Numerous degenerative disorders adversely affect RGCs, with injury to their axons resulting in vision loss or blindness. However, there has been a lack of success in the development of replacement strategies for RGCs due to obstacles such as the long-distance outgrowth of RGC axons and successful pathfinding towards post-synaptic targets.

In part, due to the inability to extend axons across long distances as well as the lack of capacity to appropriately respond to extrinsic guidance cues to regulate this outgrowth, the ability to serve as a model of retinal development is limited, as well as their utility for cell replacement therapies. As such, a need exists for the development of an in vitro system that facilitates differentiation of retinal organoids in a manner that closely mimics the spatial and temporal development of RGCs. This would provide a superior model of RGC development, facilitating applications of hPSC-derived RGCs for disease modeling, drug screening, as well as cell replacement.

As disclosed herein, retinocortical assembloids provide a natural target and an environment in which RGC axonal outgrowth can be significantly increased. Assembloids were generated by fusing retinal organoids (ROs) with cortical organoids (COs) for short term (1 week) or long term (100 days). Results provided herein indicates that RGCs extended neurites into COs as soon as 3 days post fusion (dpf), correlating with a significant increase in RO area and proliferation at 3, 5 and 7 dpf compared to ROs cultured alone. Long term assembloids allowed for significantly more RGCs surviving compared to ROs alone. Finally, RGC axons display pathfinding abilities by avoiding ventricularlike zones within cortical organoids. Results of this study demonstrate that the in vivo environment likely modulates RGC neurite outgrowth. As such, these results will facilitate the eventual use of hPSC-derived RGCs for cell replacement, in vitro disease modeling and pharmaceutical screening.

Embodiments disclosed herein include a three-dimensional tissue composition having a retinal organoid and a cortical organoid. Additional embodiments include an in vitro method of producing a three-dimensional tissue composition, comprising the steps of differentiating human pluripotent stem cells into a population retinal cells and a population of cortical cells; and allowing retinal organoids to fuse with cortical organoids. The three-dimensional tissue composition may be a retina-cortical assembloid.

Further embodiments include a method for screening for neuropsychiatric or neurological diseases, comprising the steps of generating a three dimensional tissue composition comprising a retinal organoid and a cortical organoid; and screening for dysregulation of spontaneous activity or defects of stimulus-induced activity in the three dimensional tissue composition.

Yet other embodiments include an organoid-machine interface having a multi-probe electrode array configured to collect electrophysiological signals from tissue; a first processor operably linked to the multi-probe electrode array; a second processor operably linked to a stimulus-generating device; and machine executable instructions configured to decode circuit response and instruct feedback stimulation to the sensory generating device.

Aspects of the present disclosure also include the following. Region specific organoids recapitulate lamination of retinal and cortical layers; Retinal and cortical organoids can be fused together to generate assembloids; RGC axons actively grow into cortical organoids mimicking in vivo architecture; Short term assembly of retino-cortical assembloids significantly increases retinal area and cell proliferation; Long term assembly of retino-cortical assembloids significantly increases RGC survival; and, RGCs display pathfinding abilities by avoiding ventricular-like zones within cortical organoids.

Differentiation protocols are well known to those of ordinary skill in the art. For example, some of the common differentiation protocols are disclosed in U.S. Pat. No. 9,752,119.

In some embodiments, the retinal ganglion cells with elongated axons produced by the method disclosed herein can be used as materials for regenerative medicine aimed at treatment of eye disease or retinal degenerative disease involving retinal ganglion cell damage in the form of, for example, a cell preparation or a cell sheet. Examples of an eye disease or a retinal degenerative disease involving retinal ganglion cell damage may include glaucomatous diseases, hereditary optic neuropathy, optic nerve hypoplasia, ischemic disorders, and retinal diseases. Specific examples may include, but are not limited to, glaucoma (e.g., glaucomatous constriction of the visual field and glaucomatous atrophy of the optic nerve), autosomal dominant atrophy of the optic nerve, Leber's hereditary optic neuropathy (Leber's disease), idiopathic optic neuritis, optic nerve hypoplasia involved with iridosteresis, optic neuromyelitis (demyelination), multiple sclerosis (demyelination), ischemic optic neuropathy, central retinal artery occlusion, branch retinal artery occlusion, central retinal vein occlusion, branch retinal vein occlusion, traumatic or drug-induced optic neuropathy, diabetic optic neuropathy, retinopathy of prematurity, and retinal detachment.

Yet in some embodiments, the retinal ganglion cells produced by the method disclosed herein can be used to screen for a protective agent for a retinal nerve, a regenerative agent for a retinal nerve, or the like. Screening can be carried out with the use of the retinal ganglion cells produced by the method described above (e.g., normal cell models), and screening can also be carried out with the use of retinal ganglion cells with the reproduced diseases or damages of the retinal nerve (e.g., optic neuropathy cell models).

### EXAMPLE

The following examples illustrate various aspects of the disclosure. It will be apparent to those skilled in the art that many modifications, both to materials and methods, can be practiced without departing from the scope of the disclosure.

Maintenance and expansion of hPSCs. Different lines of hPSCs were utilized in this study, including those with or without an RGC-specific fluorescent reporter. hPSCs were initially maintained in an undifferentiated state as previously described (see e.g., Ohlemacher, S. K., Iglesias, C. L., Sridhar, A., Gamm, D. M. & Meyer, J. S. Generation of highly enriched populations of optic vesicle-like retinal cells from human pluripotent stem cells. CURRENT PROTOCOLS IN STEM CELL BIOLOGY 32, 1h.8.1-20; see also Fligor C M, Huang K C, Lavekar S S, VanderWall K B, Meyer J S (2020), Differentiation of retinal organoids from human pluripotent stem cells, METHODS CELL BIOL 159:279-302). Briefly, cells were maintained in mTeSR1 medium on a Matrigel substrate. Upon reaching approximately 70% confluency, cells were mechanically passaged with dispase (2 mg/ml) and split at a ratio of 1:6, with passaging of cells occurring every 4-5 days.

Differentiation of organoids from hPSCs. For retinal organoids, hPSCs were differentiated to a retinal lineage following previously established protocols (Fligor et al. 2020). Briefly, embryoid bodies (EB) were generated by lifting hPSCs from Matrigel-coated wells using dispase (2 mg/mL). EBs were maintained in suspension and gradually transitioned to a chemically defined neural induction medium (NIM), which consisted of DMEM/F12 (1:1), N2 supplement, MEM non-essential amino acids, heparin (2 ug/mL) and PSA. After 6 days, 1.5nM of BMP4 was added to encourage retinal lineage differentiation. After 8 days, the EBs were plated onto 6-well plates with 10% FBS to ensure adhesion. Half media changes were performed on days 9 and 12 with a full media change occurring on day 15. After 16 days of differentiation, cell aggregates were mechanically lifted and kept in suspension in Retinal Differentiation Medium (RDM), which consisted of DMEM/F12 (3:1), B27 supplement, MEM non-essential amino acids, and PSA. Retinal organoids containing presumptive RGCs were maintained in this medium until experimental time points indicated.

Cortical organoid differentiation was very similar to retinal organoid differentiation. The only differences in the process was exclusion of BMP4 at 6 days of differentiation and EBs were plated on laminin coated plates after 8 days of differentiation.

Thalamic organoids were differentiated following a previously published protocol (Park et al.). Briefly, hPSCs were dissociated to single cells using Accutase. Single cells were resuspended in induction media (DMEM-F12, 15% KSR, 1% MEM-NEAA, 1% Glutamax, 1% PSA and 100 mM b-Mercaptoethanol, 100 nM LDN-193189, 10 mM SB-431542, 4 mg/ml Insulin, 5% heat-inactivated FBS, and 50 mM Y27632) and aggregated in ultra-low-attachment 96-well plates at a density of 7 k cells/well. Half media changes were performed every other day. After 8 days, aggregates were transferred to spinning culture (80 rpm/min) in 24-well low attachment plates and maintained in patterning media (DMEM-F12, 0.15% Dextrose, 100 mM b-Mercaptoethanol, 1% N2 supplement, 1% PSA, 2% B27 supplement minus vitamin A, 30 ng/ml BMP7 and 1 mM PD325901). Media was changed every other day until day 16 when differentiation media (1:1 mixture of DMEM-F12 and Neurobasal media, 0.5% N2 supplement, 1% B27 supplement, 0.5% MEM-NEAA, 1% Glutamax, 0.025% Insulin, 50 mM b-Mercaptoethanol, and 1% PSA, 20 ng/ml BDNF and 200 mM ascorbic acid) with media changes every other day until day 25, with media changes every four days thereafter.

Fusion of Assembloids. Organoid fusion was performed at 50 days of differentiation. A single BRN3:TdTomato positive retinal organoid was placed into a 1.5 mL Eppendorf tube with a single cortical organoid and 500 uL of RDM media. 3 days after assembly fused assembloids were transferred to a single well of a low attachment 24 well plate for further development using RDM media supplemented with 10% FBS, 1× Glutamax and 100 uM Taurine. Media was changed every 2-3 days. Triassmbloids were assembled in transwells to allow for more control of the position of organoids. 3 days after assembly, triassembloids were maintained as outlined above.

Immunocytochemistry and Imaging. For cryostat sectioning, retinal organoids were fixed with 4% paraformaldehyde, washed 3× in PBS, and then equilibrated in a 20% and then 30% sucrose solution overnight at 4° C. Once reaching equilibrium, organoids were embedded in OCT and frozen on dry ice and sections were cut at 11 μm thickness. Similarly, RGCs grown on coverslips were fixed in 4% paraformaldehyde and washed 3× in PBS before staining.

Immunocytochemical staining of samples was performed as previously described. Briefly, permeabilization was performed in 0.2% Triton X-100 for 10 minutes and samples were then blocked in 10% donkey serum for one hour at room temperature. Primary antibodies were diluted as indicated (Table 51) in 0.1% Triton X-100 and 5% donkey serum and applied overnight at 4° C. The following day, samples were washed in PBS and blocked with 10% donkey serum for 10 minutes. Secondary antibodies were diluted 1:1000 in 0.1% Triton X-100 and 5% donkey serum and applied for one hour at room temperature. Finally, cells were washed with PBS and mounted onto slides for imaging.

Quantification and statistical analysis. The number of cells expressing unique retinal markers was quantified in cryostat sections of retinal organoids at indicated timepoints. Multiple biological replicates were obtained at each time point (n=3) and Image-J was used to quantify the expression of each marker as indicated in results. One-Way ANOVA statistical analyses at 95% confidence (post hoc Tukey) was performed, excluding outliers, to determine significant differences in cell counts over time. Statistical significances were determined based on a p value less than 0.05. To analyze retinal organoid-derived RGCs, mCherry- or tdTomato-positive RGCs were quantified, and the co-expression of these reporters with RGC or other retinal cell type markers was quantified using the Image-J cell counter. Four distinct regions of at least three coverslips were imaged and quantified, with these experiments repeated with at least three different groups of cells. The percentage of mCherry-positive cells colocalizing with retinal cell type markers and the standard error of the mean was quantified.

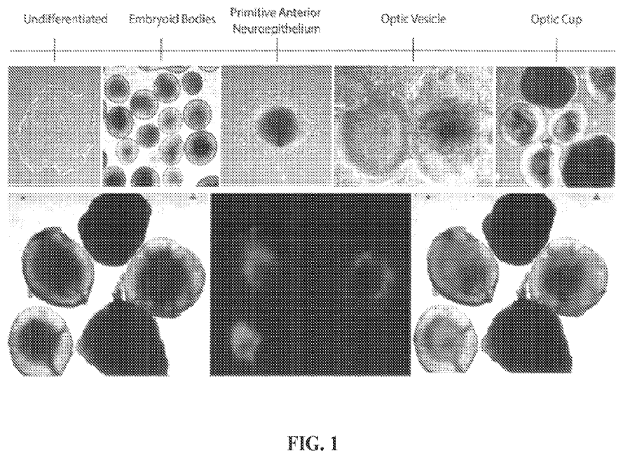

Referring now to FIG. 1, differentiation of retinal and cortical organoids is shown. For example, hPSCs were differentiated following established protocols that generates two populations of organoids. Retinal organoids are identified by their bright outer ring as well as a BRN3:TdTomato red fluorescent reporter. Cortical organoids develop along side retinal organoids and contain neural rosette structures throughout.

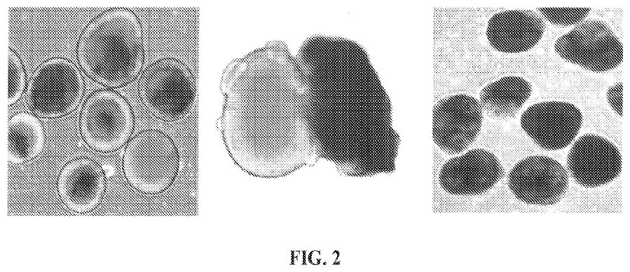

Referring now to FIG. 2, generation of retina-cortical assembloids is shown. RGCs could be readily identified by TdTomato expression observed in the inner layers of each organoid, defining the presumptive retinal ganglion cell layer. To create retina-cortical assembloids retinal organoids are allowed to fuse with cortical organoids for three days. The BRN3:TdTomato reporter allows for visualization of RGC neurites extending into cortical organoids. Generation of retinal-cortical assembloids more accurately models in vivo architecture of the human diencephalon, composed of the forebrain and developing eyecup.

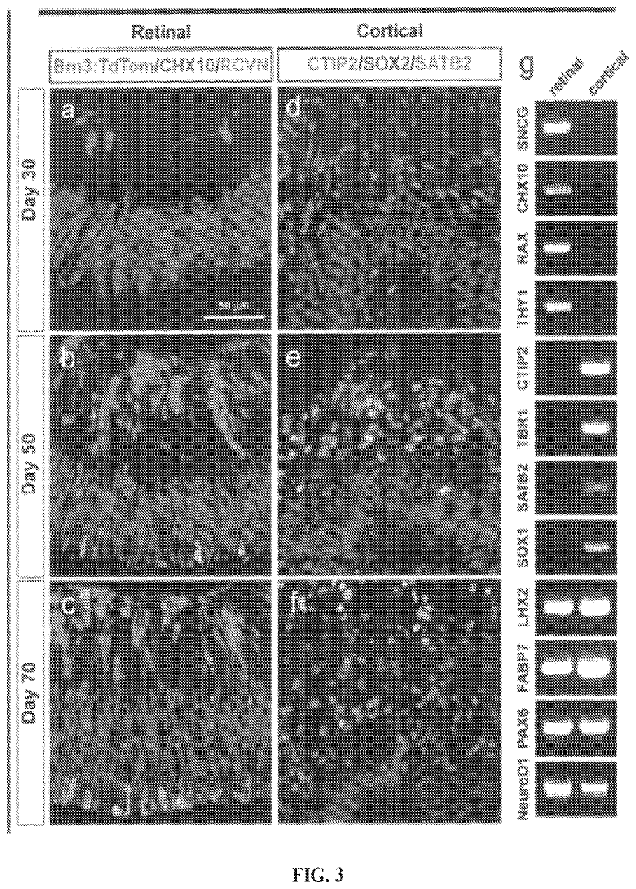

Generation of two genetically distinct populations of organoids is shown. RGCs begin to develop in the innermost layer of retinal organoids around 30 days of differentiation (FIGS. 3A-3C), followed by the development of a distinctly separate photoreceptor layer by 70 days of differentiation (FIGS. 3D-3F). Cortical organoids contain rosettes enriched with progenitors, similar to the ventricular zone. Earlier born CTIP2+ neurons reside outside of the ventricular zone and later born SATB2+ neurons begin to migrate to a separate outer layer within cortical organoids. Both organoid populations are of neural origin, however, retinal organoids express retinal markers while cortical organoids express cortical markers (FIG. 3G).

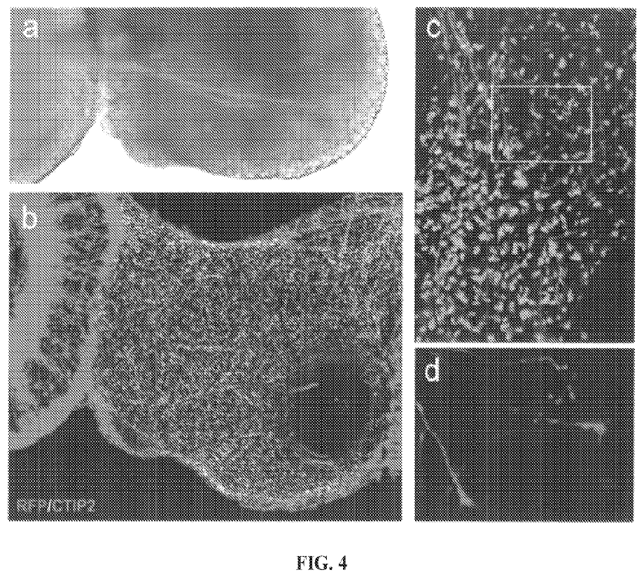

Retina-cortical assembloids mimic in vivo architecture. Retinal and cortical organoids can be fused to form retina-cortical assembloids. BRN3:TdTomato RGCs extend axons into cortical organoids (FIGS. 4A-4B). Growth cones at the leading edge of RGC axons confirms RGCs are actively growing into cortical neurons, suggesting the cortical environment supports RGC outgrowth (FIGS. 4C-4D).

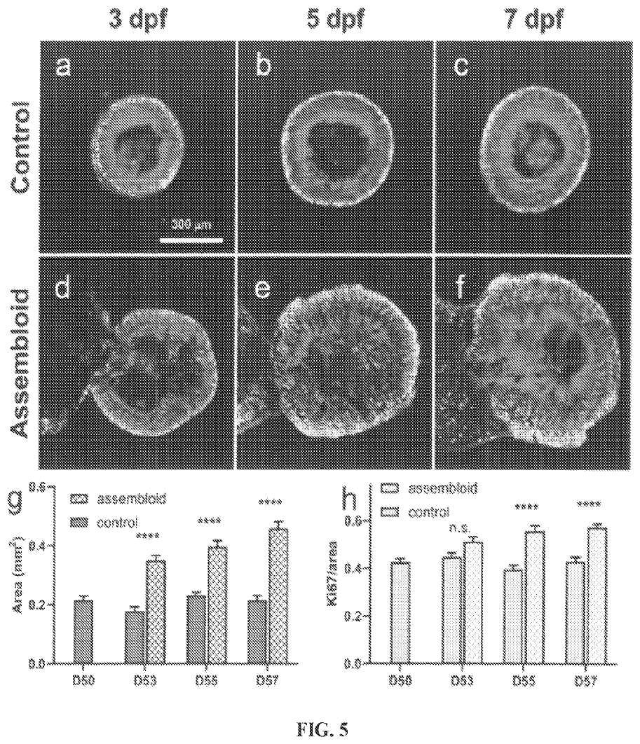

Assembloids significantly increase retinal area and proliferation. Retinal organoids grown alone were compared to retinal organoids fused to cortical organoids after 3, 5, and 7 days post fusion (dpf) (FIGS. 5A-5F). After 3 days of fusing with cortical organoids (53 days total growth) retinal area of retina-cortical assembloids was significantly increased compared to control retinal organoids (FIG. 5G). (Retina-cortical assembloids displayed a significant increase in cell proliferation by 55 days of total differentiation when compared to controls (FIG. 511).

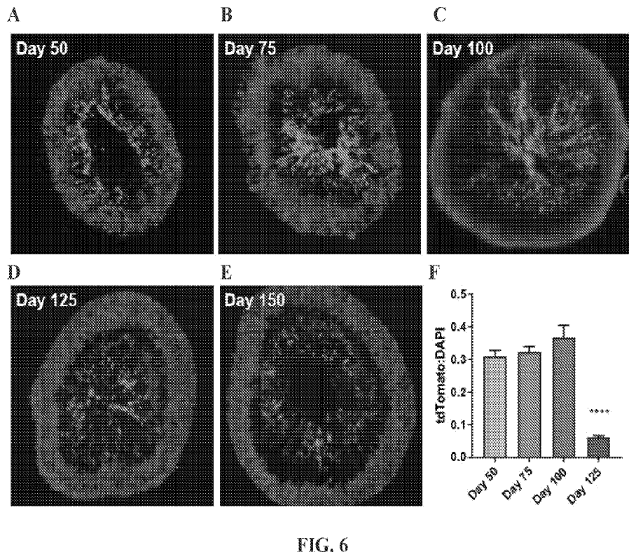

Results show that long term retinal organoids maintain RGC populations. BRN3:TdTomato expression begins to decrease in retinal organoids after 100 days in culture (FIGS. 6A-6F).

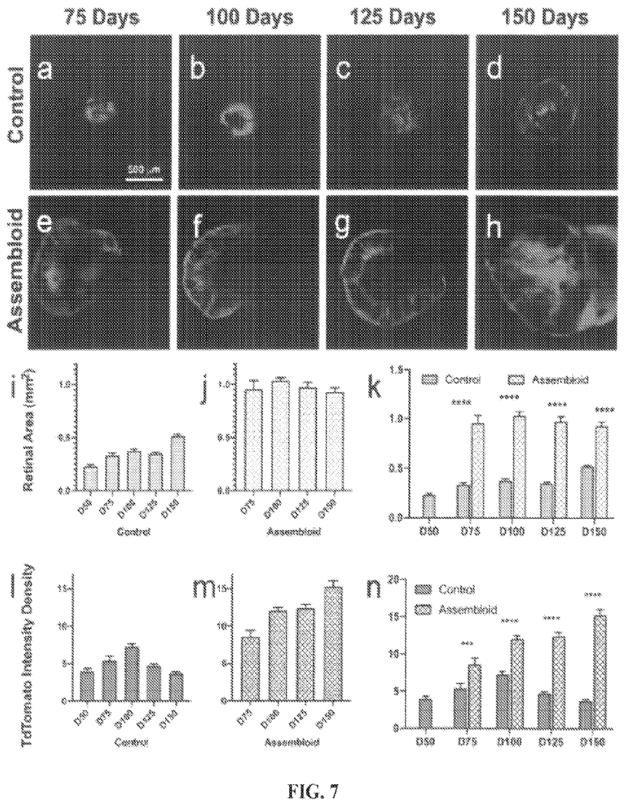

The long-term retina-cortical assembloids maintain RGC populations. Retinal organoids were maintained up to 150 days in culture and compared to age matched retina-cortical assembloids (FIGS. 7A-71I). Retinal area was significantly increased in assembloids (FIGS. 7I-7K). BRN3:TdTomato expression begins to decrease in control organoids after 100 days in culture, while expression is significantly increased in assembloids and continues to increase over time (FIGS. 7L-7N).

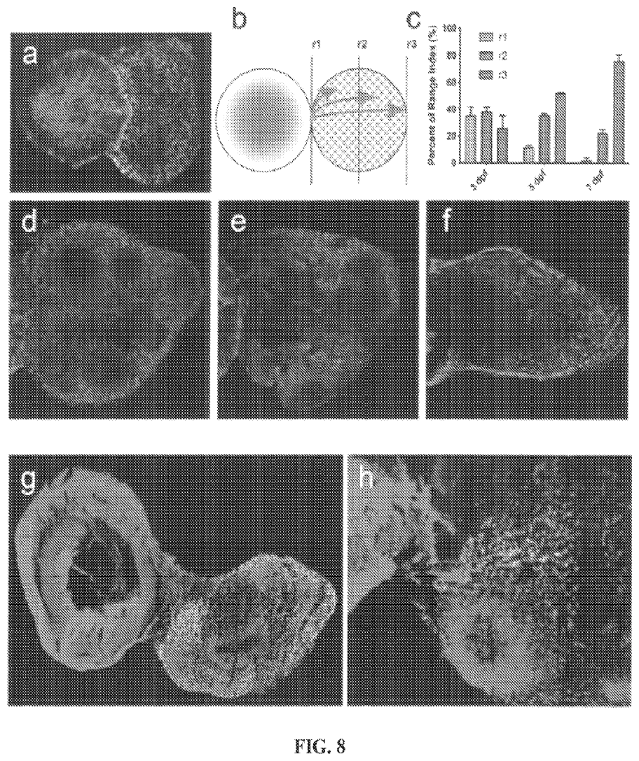

FIGS. 8A-81I show that RGC axons actively grow into assembloids and avoid ventricular zone. Retinal organoids expressing BRN3:TdTomato are fused to CTIP2+ cortical organoids at 50 days of differentiation (FIG. 8A). FIG. 8B shows schematic view of method for quantifying axonal outgrowth and FIG. 8C provides quantification of range index at 3, 5, and 7 days post fusion (dpf). Representative images of outgrowth at 3 dpf (d) 5 dpf (e) and 7 dpf (f) (FIGS. 8D-8F). RGC axons display outgrowth abilities by extending into cortical organoids. RGC axons also display pathfinding abilities by avoiding SOX2+ ventricular-like zones within cortical organoids (FIGS. 8G-8H).

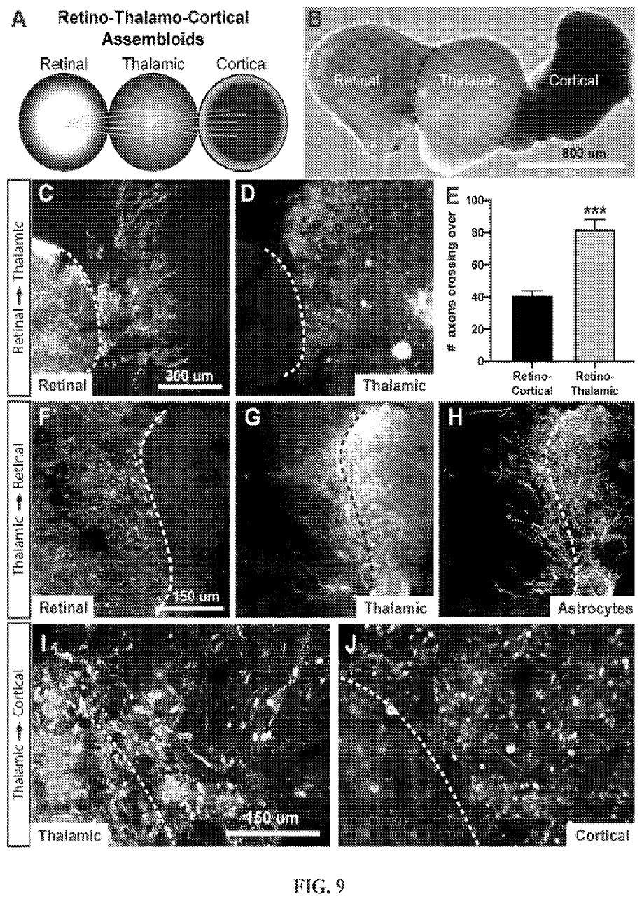

FIGS. 9A-9J show visual pathway reconstruction with retinal, thalamic, and cortical organoids. Retinal, thalamic and cortical organoids were fused together for to generate Retino-Thalamo-Cortical triassembloids (FIG. 9A). Fluorescent reporters were used to identify various organoids and their projections with retinal organoids expressing a BRN3:tdTomato reporter and thalamic organoids expressing a GFP reporter (FIG. 9B). Within one week following the fusion of organoids to form assembloids, tdTomato-expressing retinal ganglion cell axons were found to robustly extend into GFP-expressing thalamic organoids (FIGS. 9C-9D). Significantly greater numbers of RGC axons had extended into thalamic organoids compared to cortical organoids at the same time point (FIG. 9E). After an additional 2 months of differentiation, GFP-expressing thalamic cells were found migrating retrogradely into retinal organoids, with these migratory cells expressing the early astrocyte marker S100β (FIGS. 9F-9H). On the other side of these assembloids, robust extension of GFP-expressing neurites was observed entering CTIP2-positive cortical organoids (FIGS. 9I-9J).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

1. A three-dimensional neural tissue composition, comprising:
an assembloid comprising two or more region-specific organoids, comprising:
at least one retinal organoid;
at least one cortical organoid;
at least one thalamic organoid; and
at least one other region-specific organoid that does not recapitulate the development of retina, cortex, or thalamus,
wherein the two or more region-specific organoids are operatively fused to form the assembloid.

2. The composition of claim 1, wherein the assembloid comprises the at least one retinal organoid and the at least one cortical organoid.

3. The composition of claim 1, wherein the assembloid comprises the at least one retinal organoid, the at least one cortical organoid, and the at least one thalamic organoid.

4. The composition of claim 1, wherein the at least one cortical organoid is fused directly to the at least one retinal organoid and/or the at least one thalamic organoid is fused directly to the at least one retinal organoid.

5. The composition of claim 1, wherein a first end of the at least one thalamic organoid is fused directly to the at least one retinal organoid and a second end of the at least one thalamic organoid is fused directly to the at least one cortical organoid.

6. The composition of claim 1, the composition further comprises retinal ganglion cells residing in the at least one retinal organoid.

7. The composition of claim 6, wherein the retinal ganglion cells (RGC) have axons extending into the at least one thalamic organoid and/or the at least one cortical organoid.

8. The composition of claim 1, the composition further comprises thalamic cells in the at least one thalamic organoid that have migrated into the at least one retinal organoid.

9. The composition of claim 1, each of the at least one retinal organoid, the at least one cortical organoid, and the at least one thalamic organoid is derived from human pluripotent stem cells.

10. The composition of claim 1, the assembloid further comprises the highly proliferative retinal ganglion cells (RGC) compared to the retinal ganglion cells (RGC) grown in the at least retinal organoid alone.

11. A method of generating retinal ganglion cells (RGC) with elongated axons, comprising:
generating the assembloid of any one of claims 1; and
isolating retinal ganglion cells (RGC).

12. The method of claim 11, further comprising:
differentiating pluripotent stem cells into at least one region-specific organoid, comprising:
at least one retinal organoid;
at least one cortical organoid;
at least one thalamic organoid; and
at least one other region-specific organoid that does not recapitulate the development of retina, cortex, or thalamus.

13. The method of claim 1, further comprising:
subjecting pluripotent stem cells to floating culture to induce differentiation into retinal progenitor cells.

14. The method of claim 11, further comprising:
developing the at least one region-specific organoid separately; and
fusing two or more region-specific organoids.

15. The method of claim 11, further comprising:
allowing axons to elongate into the at least one region-specific organoid other than the retinal organoid.

16. The method of claim 1, wherein the region-specific organoids are developed separately for about 50 days prior to fusion and fused for about 5-10 days.

17. The method of claim 11, the pluripotent stem cells comprise embryonic stem cells and/or induced pluripotent stem cells, wherein the pluripotent stem cells are derived from a human or an animal.

18. A therapeutic composition comprising a plurality of retinal ganglion cells with elongated axons obtained by the method according to laim 1 and/or the composition according to claim 1.

19. A method for treating a retinal degenerative disease, comprising:
administering to a subject a therapeutically effective amount of the therapeutic composition according to claim 18, wherein the subject is diagnosed with a retinal degenerative disease or a related condition thereof.

20. The method of claim 19, the retinal degenerative disease or a related condition thereof comprise age-related macular degeneration and retinitis pigmentosa.
